(19) United States
(12) Patent Application Publication
(10) Pub. No.: US 2025/0107798 A1
(43) Pub. Date: Apr. 3, 2025

(54) SYSTEMS AND METHODS FOR PERFORMING TENSIONABLE KNOTLESS CARDIAC VALVE PROCEDURES

(71) Applicant: ARTHREX, INC., Naples, FL (US)

(72) Inventors: Andrew Christian Petry, Naples, FL (US); Peter J. Dreyfuss, Naples, FL (US); Paul DiGiorgi, Fort Myers, FL (US); Daniel T. Engelman, Longmeadow, MA (US)

(21) Appl. No.: 18/420,100

(22) Filed: Jan. 23, 2024

Related U.S. Application Data
(60) Provisional application No. 63/586,097, filed on Sep. 28, 2023.

(51) Int. Cl. A61B 17/04; A61B 17/00

(52) U.S. Cl. CPC A61B 17/0487; A61B 2017/00743; A61B 2017/00982

(57) ABSTRACT

Systems and methods are provided for performing tensionable knotless cardiac valve procedures. A locking ferrule that includes a one-way locking mechanism may be utilized as part of the tensionable knotless cardiac valve procedure for tensioning and locking one or more strands of suture. In other implementations, the one-way locking mechanism may be integrated as part of a prosthetic device or other surgical device that can be utilized in combination with the prosthetic device.

### CROSS-REFERENCE TO RELATED APPLICATIONS

This application claims the benefit of U.S. Provisional Application No. 63/586,097, which was filed on Sep. 28, 2023 and is incorporated herein by reference in its entirety.

### BACKGROUND

This disclosure relates to the field of surgery, and more particularly to systems and methods for performing cardiac valve procedures.

The human heart includes multiple valves. The valves open and close as the heart pumps to ensure that blood flows in the proper direction. The structure and/or function of a heart valve can become impaired due to various factors such as age, genetic defects, infection, or other chronic conditions, thereby necessitating its surgical repair or replacement.

### SUMMARY

This disclosure relates to systems and methods for performing tensionable knotless cardiac valve procedures. A locking ferrule that includes a one-way locking mechanism may be utilized as part of the tensionable knotless cardiac valve procedure for tensioning and locking one or more strands of suture.

An exemplary locking ferrule for performing a cardiac valve surgical procedure may include, inter alia, a body including an outer diameter wall, an inner diameter wall, and a cannulation that is circumscribed by the inner diameter wall. A plurality of locking barbs extend into the cannulation and are configured to lock a suture relative to the body after the suture has been received through the cannulation.

An exemplary surgical method may include, inter alia, passing a suture through a tissue of a heart valve, passing the suture through a prosthetic device, shuttling the prosthetic device along the suture and into the heart valve, loading the suture through a cannulation of a locking ferrule, tensioning the suture in a first direction, and locking the suture within the cannulation to prevent movement of the suture in a second direction.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates a locking ferrule that includes a one-way locking mechanism.

FIG. 2 is an end view of the locking ferrule of FIG. 1.

FIG. 3 is a cross-sectional view through section 3-3 of FIG. 1.

FIG. 4 is a cross-sectional view through section 4-4 of FIG. 2.

FIG. 5 schematically illustrates loading a suture through a locking ferrule.

FIG. 6 schematically illustrates locking a suture relative to a locking ferrule.

FIG. 7 illustrates an angled locking ferrule.

FIG. 8 illustrates a slotted locking ferrule.

FIGS. 9, 10, 11, 12, and 13 schematically illustrate a surgical method for performing a heart valve repair or replacement.

FIGS. 14, 15, and 16 illustrate an exemplary inserter device for implanting a plurality of locking ferrules relative to a prosthetic device.

FIG. 17 illustrates an exemplary prosthetic device that can include one or more pre-loaded locking ferrules.

FIG. 18 illustrates an exemplary surgical pledget that can include one or more pre-loaded locking ferrules.

FIG. 19 illustrates another exemplary prosthetic device.

### DETAILED DESCRIPTION

This disclosure relates to systems and methods for performing tensionable knotless cardiac valve procedures. A locking ferrule that includes a one-way locking mechanism may be utilized as part of the tensionable knotless cardiac valve procedure for tensioning and locking one or more strands of suture. These and other features of this disclosure are described in further detail below.

An exemplary locking ferrule for performing a cardiac valve surgical procedure may include, inter alia, a body including an outer diameter wall, an inner diameter wall, and a cannulation that is circumscribed by the inner diameter wall. A plurality of locking barbs extend into the cannulation and are configured to lock a suture relative to the body after the suture has been received through the cannulation.

In any further embodiment, the plurality of locking barbs are integral features of the body.

In any further embodiment, each of the plurality of locking barbs includes a pointed tip.

In any further embodiment, the body extends along a longitudinal axis between a proximal end and a distal end.

In any further embodiment, each of the plurality of locking barbs is angled in a direction toward the proximal end.

In any further embodiment, the plurality of locking barbs each protrude inwardly from the inner diameter wall.

In any further embodiment, the plurality of locking barbs are arranged in at least a first row and a second row.

In any further embodiment, a first portion of the plurality of locking barbs of the first row are staggered relative to a second portion of the plurality of locking barbs of the second row.

In any further embodiment, the body of the locking ferrule is angled.

In any further embodiment, a slot is formed through the body of the locking ferrule.

An exemplary surgical method may include, inter alia, passing a suture through a tissue of a heart valve, passing the suture through a prosthetic device, shuttling the prosthetic device along the suture and into the heart valve, loading the suture through a cannulation of a locking ferrule, tensioning the suture in a first direction, and locking the suture within the cannulation to prevent movement of the suture in a second direction.

In any further embodiment, a locking barb of the locking ferrule locks the suture relative to a body of the locking ferrule.

In any further embodiment, the locking barb protrudes inwardly from an inner diameter wall of the locking ferrule.

In any further embodiment, the locking barb engages a thickened section of the suture to prevent its movement in the second direction.

In any further embodiment, loading the suture through the cannulation includes pulling the suture through the cannulation in the first direction with a suture loader.

In any further embodiment, the method includes moving an eyelet of the suture loader in the second direction through the cannulation prior to pulling the suture through the cannulation.

In any further embodiment, pulling the suture through the cannulation includes pulling a thinned section of the suture through the cannulation.

In any further embodiment, the locking ferrule is pre-loaded within a pocket of the prosthetic device.

In any further embodiment, passing the suture through the prosthetic device and loading the suture through the cannulation are performed as a single step.

In any further embodiment, the prosthetic device is an annuloplasty ring or a replacement heart valve.

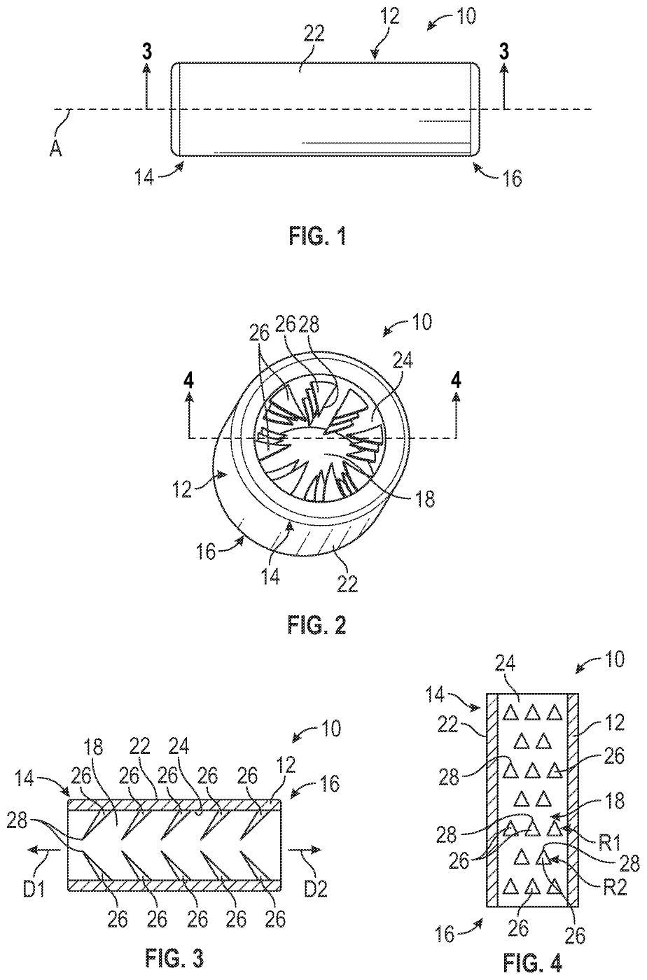

FIGS. 1-6 illustrate an exemplary locking ferrule 10 that can be used when performing tensionable knotless cardiac valve procedures. For example, the locking ferrule 10 could be utilized during surgical methods for repairing or replacing a human heart valve (e.g., aortic, mitral, tricuspid, or pulmonary).

The locking ferrule 10 may include a body 12 that extends along a longitudinal axis A between a proximal end 14 and a distal end 16. The body 12 may be tubular shaped and may be constructed from either metallic materials or plastic materials. However, the specific size, shape, and material make-up of the body 12 are not intended to limit this disclosure.

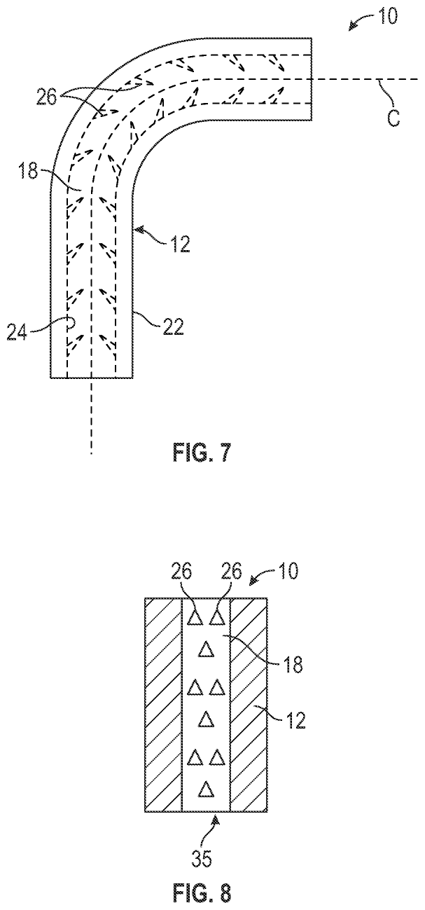

In an embodiment, the body 12 is substantially straight along the longitudinal axis A. In another embodiment, the body 12 may be angled (see FIG. 7) to enable portions of the locking ferrule 10 to face away from leaflets of the valve being repaired, for example.

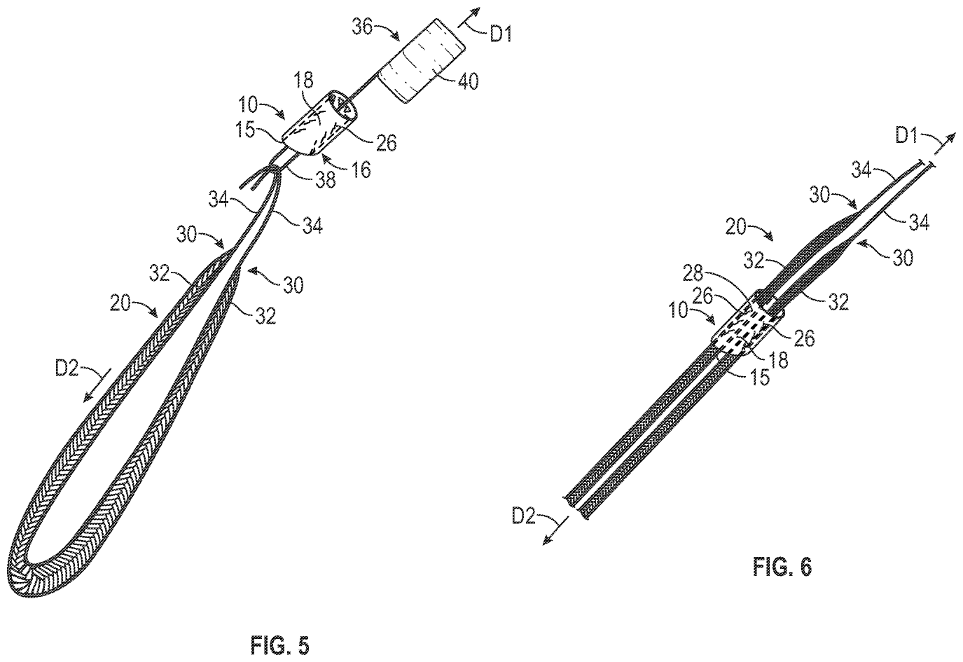

A cannulation 18 may extend through the body 12 and may establish an internal passageway for accommodating one or more strands of suture 20 (see, for example, FIGS. 5 and 6). The cannulation 18 may extend across an entire length of the body 12 and thus extends from the proximal end 14 to the distal end 16. In an embodiment, the longitudinal axis A may bisect the cannulation 18. In another embodiment, such as when the body 12 is angled, the cannulation 18 may extend along a non-linear centerline C (see FIG. 7).

The body 12 may include an outer diameter wall 22 and an inner diameter wall 24. The outer diameter wall 22 may be smooth or could alternatively include threads, barbs, or other features for facilitating bone fixation. The inner diameter wall 24 may circumscribe the cannulation 18. In some embodiments, the cannulation 18 may taper in a direction toward the distal end 16 and is therefore narrower within the distal end 16 compared to within the proximal end 14.

A plurality of locking barbs 26 may protrude inwardly from the inner diameter wall 24. The locking barbs 26 may therefore occupy at least a portion of the open space of the cannulation 18. In an embodiment, the locking barbs 26 are integrally formed (e.g., molded) features of the body 12 of the locking ferrule 10. The locking barbs 26 may be provided along an entire length of the cannulation 18 or at only select portions thereof. The locking barbs 26 may be either rigid or flexible structures.

The locking barbs 26 may be arranged in multiple rows along the length of the cannulation 18. For example, the locking barbs 26 may be arranged in a least a first row R1 and a second row R2 (see FIG. 4). In an embodiment, the locking barbs 26 of the second row R2 are staggered relative to the locking barbs 26 of the first row R1 (see FIG. 4).

Each locking barb 26 may include a sharp or pointed tip 28, and each locking barb 26 may be angled in a direction toward the proximal end 14. The locking barbs 26 may therefore establish a one-way locking mechanism that permits one or more sutures 20 to pass through the cannulation in a first direction D1 while preventing the suture(s) 20 from being tensioned or otherwise moved in a second direction D2. The staggered relationship of the rows of locking barbs 26 may provide for maximum engagement with the suture 20 one it is passed through the cannulation 18.

Referring now primarily to FIGS. 5 and 6, with continued reference to FIGS. 1-4, one or more sutures 20 may be passed through the cannulation 18. The suture 20 may be FiberWire®, FiberTape®, or any other suitable suture product. FiberWire® and FiberTape® are suture products marketed and sold by Arthrex, Inc. However, other suture products could be utilized for the suture 20 within the scope of this disclosure. The size, shape, and type of suture utilized in conjunction with the locking ferrule 10 are not intended to limit this disclosure.

The suture 20 may include a varying thickness. The suture 20 may therefore include one or more tapered regions 30 where the suture 20 transitions between a thickened section 32 and a thinned section 34. The thinned sections 34 can facilitate passing the reinforcement suture 20 through the cannulation 18, such as via a suitable suture loader 36 that can be provided as part of a suture locking system that includes the locking ferrule 10, for example, and the thickened sections 32 can provide greater surface area for the locking barbs 26 to engage in order to sufficiently lock the reinforcement suture 20 relative to the locking ferrule 10.

In an embodiment, the thickened sections 32 of the reinforcement suture 20 are about twice as thick as the thinned sections 34. However, other ratios (e.g., 1.5:1, 3:1, etc.) between the relative thicknesses (e.g., outer diameters) of the thickened sections 32 and the thinned sections 34 are contemplated within the scope of this disclosure.

The one or more sutures 20 may be passed through the cannulation 18 of the locking ferrule 10 using the suture loader 36. An eyelet 38 of the suture loader 36 may be passed through the cannulation 18 (e.g., by inserting the eyelet 38 at the distal end 16 of the body 12 and then moving the suture loader 36 in the second direction D2). One or more thinned sections 34 of the suture 20 may then be loaded through the eyelet 38. The suture loader 36 may then be pulled, via a handle 40, in the first direction D1 to pass the suture 20 through the cannulation 18.

In another embodiment, a slot 35 (see FIG. 8) may be formed through the body 12 of the locking ferrule 10 for facilitating the loading of the suture 20 into the cannulation 18. The slot 35 may be tapered to allow the suture 20 to more easily pass into the cannulation 18 without easily backing out, and/or the body 12 could be crimped to reduce the width of the slot 35 after positioning suture 20 within the cannulation 18.

Once the suture 20 has been passed through the cannulation 18, the suture 20 may be tensioned in the first direction D1 to lock the suture 20 relative to the locking ferrule 10. The locking barbs 26 prevent the suture 20 from backing up or otherwise moving in the second direction D2. The pointed tips 28 of the locking barbs 26 may interdigitate with one or more of the thickened sections 32 to lock the suture 20 and prevent it from moving in the second direction D2.

Although shown as locking a single folded suture 20 in the above implementations, the locking ferrule 10 could be configured to receive and lock multiple strands of suture and/or sutures of varying shapes and sizes.

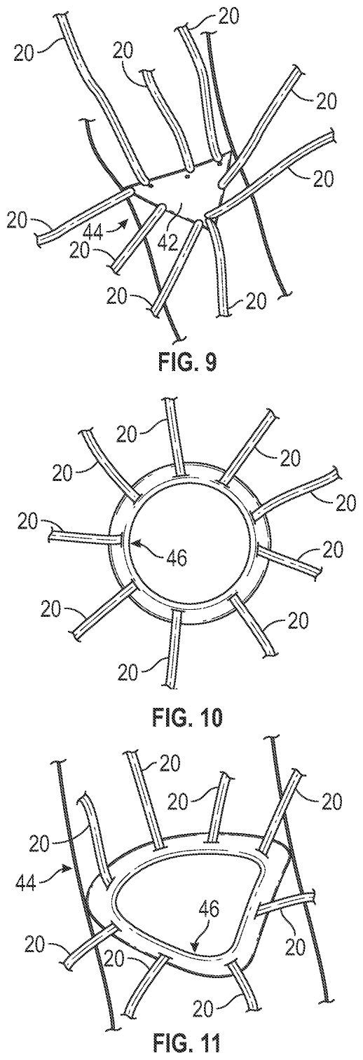

The locking ferrules 10 described above may be utilized to tension and knotlessly fixate one or more sutures 20 as part of various surgical methods. FIGS. 9-13 schematically illustrate one such surgical method for performing a heart valve repair or replacement.

In an embodiment, the surgical method is performed as an arthroscopic procedure by working through various arthroscopic portals. However, the exemplary surgical method could alternatively be performed as an open procedure within the scope of this disclosure.

A plurality of sutures 20 may be passed through a tissue 42 (e.g., muscle) associated with a heart valve 44 that includes a defect (see FIG. 9). The sutures 20 may subsequently be passed through a prosthetic device 46 (see FIG. 10). The prosthetic device 46 may be an annuloplasty ring or a ring portion of a replacement valve, for example. The prosthetic device 46 may next be shuttled down over the sutures 20 to position the prosthetic device 46 at a desired location within the heart valve 44 (see FIG. 11).

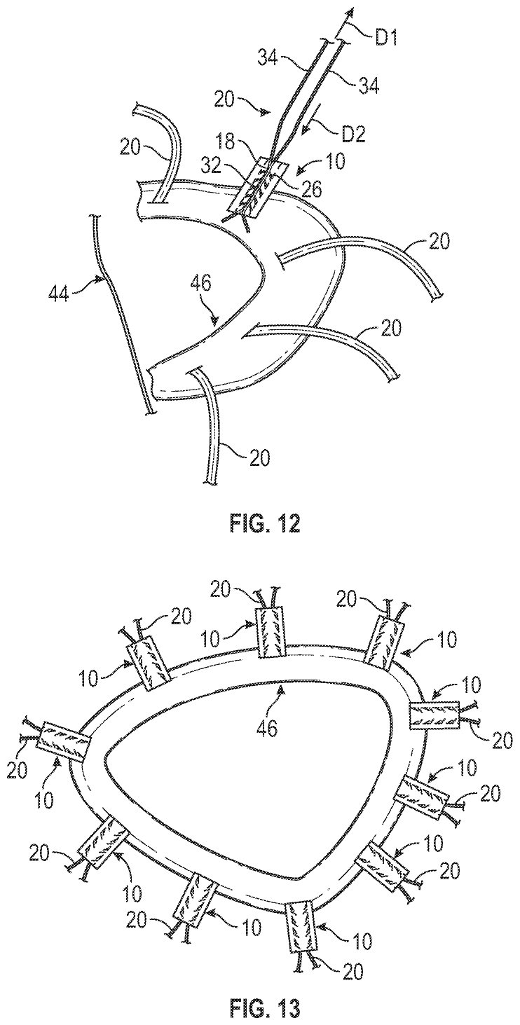

The surgical method may then proceed by shuttling the sutures 20 through a plurality of locking ferrules 10. For example, as shown in FIG. 12, after loading the thinned sections 34 of a first of sutures 20 through the cannulation 18 of one of the locking ferrules 10 (e.g., in the manner shown in FIGS. 5-6), the suture 20 may be tensioned in the direction D1 to allow the locking ferrule 10 to slide down the suture 20 in the direction D2 and be received in abutting engagement with the prosthetic device 46. Further tension of the suture 20 in the direction D1 allows the pointed tips 28 of the locking barbs 26 to interdigitate with the thickened sections 32 of the suture 20 to lock the suture 20 and prevent sliding in the second direction D2. This process may be repeated for each previously passed suture 20 in order to knotlessly fixate the prosthetic device 46 within the heart valve 44 using a plurality of the locking ferrules 10 (see FIG. 13). The excess length of the sutures 20 that extend outside of the cannulation 18 of each locking ferrule 10 may be removed (e.g., cut) once a desired level of tensioning and locking has been achieved.

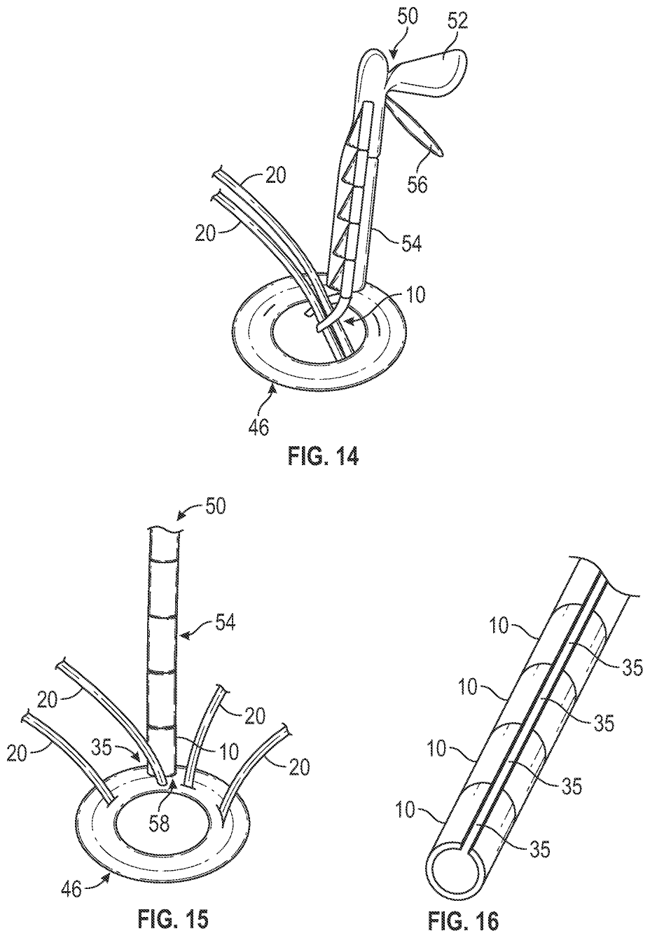

FIGS. 14-16 illustrate an inserter device 50 that can be used to implant each locking ferrule 10 relative to the prosthetic device 46. The inserter device 50 may include a handle 52 and a delivery shaft 54 that extends from the handle 52. A plurality of the locking ferrules 10 may be preloaded into the delivery shaft 54. A trigger 56 of the handle 52 may be squeezed or otherwise actuated to eject the locking ferrules 10 from the delivery shaft 54 one at a time. For example, the trigger 56 may be operably connected with a driver member (not shown) that extends through the delivery shaft 54 and is operable to deploy each of the locking ferrules 10 from the delivery shaft 54 one at a time.

In use, a tip 58 (see FIG. 15) of the delivery shaft 54 may be moved into engagement with the prosthetic device 46, and the delivery shaft 54 may then be maneuvered to position one of the previously passed sutures 20 within a slot 35 of a first of the locking ferrules 10. The locking ferrule 10 may then be ejected from the delivery shaft 54 by squeezing the trigger 56 and subsequently crimped to the suture 20 to secure the locking ferrule 10 relative to the prosthetic device 46. The suture 20 may then be tensioned and locked in the manner shown in FIGS. 5 and 6. This process may be repeated in order to knotlessly fixate the prosthetic device 46 using the inserter device 50.

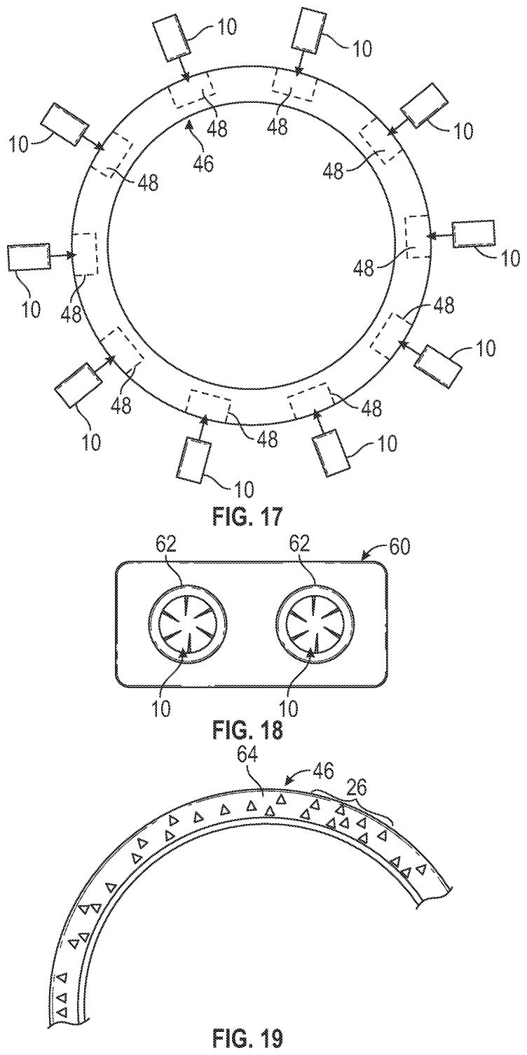

In the above embodiments, the locking ferrules 10 are separately connected to the prosthetic device 46. However, the locking ferrules 10 could alternatively be pre-loaded into pockets 48 formed in the prosthetic device 46 (see FIG. 17). In such an embodiment, each suture 20 may be passed through the prosthetic device 46 and one of the locking ferrules 10 in a single step.

In yet another embodiment, one or more locking ferrules 10 may be pre-loaded into a surgical pledget 60 (see FIG. 18) that can be used to fixate the prosthetic device 46 to tissue. One locking ferrule 10 may be received within each through-hole 62 of the surgical pledget 60.

In yet another embodiment, the locking barbs 26 for establishing the one-way locking mechanism may be integrally formed into an exterior surface 64 of the prosthetic device 46 (see FIG. 19), thereby eliminating the need for providing a separate locking ferrule. In such an embodiment, each suture 20 may be passed through the prosthetic device 46, tensioned, and locked as part of a single method step.

The systems and methods of this disclosure may be utilized to perform tensionable knotless valve repair or replacement procedures. The locking ferrules provide a one-way mechanism for tensioning and retensioning suture(s) at various points of the procedure, including subsequent to implantation of a prosthetic device, thus providing numerous advantages over prior techniques.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A locking ferrule for performing a cardiac valve procedure, comprising:
a body including an outer diameter wall, an inner diameter wall, and a cannulation that is circumscribed by the inner diameter wall; and
a plurality of locking barbs extending into the cannulation and configured to lock a suture relative to the body after the suture is passed through the cannulation.

2. The locking ferrule as recited in claim 1, wherein the plurality of locking barbs are integral features of the body.

3. The locking ferrule as recited in claim 1, wherein each of the plurality of locking barbs includes a pointed tip.

4. The locking ferrule as recited in claim 1, wherein the body extends along a longitudinal axis between a proximal end and a distal end.

5. The locking ferrule as recited in claim 4, each of the plurality of locking barbs is angled in a direction toward the proximal end.

6. The locking ferrule as recited in claim 1, wherein the plurality of locking barbs each protrude inwardly from the inner diameter wall.

7. The locking ferrule as recited in claim 6, wherein the plurality of locking barbs are arranged in at least a first row and a second row.

8. The locking ferrule as recited in claim 7, wherein a first portion of the plurality of locking barbs of the first row are staggered relative to a second portion of the plurality of locking barbs of the second row.

9. The locking ferrule as recited in claim 1, wherein the body is angled.

10. The locking ferrule as recited in claim 1, comprising a slot formed through the body.

11. A surgical method, comprising:
passing a suture through a tissue of a heart valve;
passing the suture through a prosthetic device;
shuttling the prosthetic device along the suture and into the heart valve;
loading the suture through a cannulation of a locking ferrule;
tensioning the suture in a first direction; and
locking the suture within the cannulation to prevent movement of the suture in a second direction.

12. The surgical method as recited in claim 11, wherein a locking barb of the locking ferrule locks the suture relative to a body of the locking ferrule.

13. The surgical method as recited in claim 12, wherein the locking barb protrudes inwardly from an inner diameter wall of the locking ferrule.

14. The surgical method as recited in claim 12, wherein the locking barb engages a thickened section of the suture to prevent its movement in the second direction.

15. The surgical method as recited in claim 11, wherein loading the suture through the cannulation includes:
pulling the suture through the cannulation in the first direction with a suture loader.

16. The surgical method as recited in claim 15, comprising:
moving an eyelet of the suture loader in the second direction through the cannulation prior to pulling the suture through the cannulation.

17. The surgical method as recited in claim 15, wherein pulling the suture through the cannulation includes:
pulling a thinned section of the suture through the cannulation.

18. The surgical method as recited in claim 11, wherein the locking ferrule is pre-loaded within a pocket of the prosthetic device.

19. The surgical method as recited in claim 18, wherein passing the suture through the prosthetic device and loading the suture through the cannulation are performed as a single step.

20. The surgical method as recited in claim 11, wherein the prosthetic device is an annuloplasty ring or a replacement heart valve.
